(19) United States
(12) Patent Application Publication
(10) Pub. No.: US 2004/0039051 A1
(43) Pub. Date: Feb. 26, 2004

(54) Dynamic platinum compounds for the treatment of cancer

(76) Inventor: Jiajiu Shaw, Ann Arbor, MI (US)

Correspondence Address: JIAJIU SHAW, Unitech Pharmaceuticals, Inc., 4220 A Varsity Dr., Ann Arbor, MI, 48108, US

(21) Appl. No.: 10/643,339

(22) Filed: Aug. 19, 2003

Related U.S. Application Data
(60) Provisional application No. 60/405,325, filed on Aug. 22, 2002.

(51) Int. Cl. C07F 15/00; A61K 31/28

(52) U.S. Cl. 514/492; 556/137

(57) ABSTRACT

This invention discloses a series of dynamic platinum compounds and method of treating cancer by administering said compound to a cancer patient.

### FIELD OF INVENTION

This invention relates to a series of dynamic platinum compounds, which can be activated and deactivated reversibly depending on the biological environment, and methods of treating cancer by said platinum compounds.

### BACKGROUND

Cisplatin, cis-diamminedichloroplatinum (commercial name is Platinol®), has been used as a chemotherapeutic agent for about twenty years since the discovery of its anti-tumor activity by B. Rosenberg. The Oct. 23, 1995 issue of Chemical & Engineering News reported, “Cisplatin was first synthesized in the 1800s, but its anticancer activity was not discovered until the 1960s . . . . In 1979, it was approved by the Food and Drug Administration (FDA) for clinical treatment of testicular and ovarian tumors and cancers of the head and neck”. The Physician's Desk Reference states that cisplatin can be used to treat testicular cancer, ovarian cancer, and bladder cancer.

As a first generation platinum drug, Cisplatin is still being widely used because of its efficacy. However, it is far from being a perfect anticancer drug. Carboplatin (Paraplatin®), was approved by the FDA as the second platinum drug. It appears to have a better therapeutic index than Cisplatin and is more widely prescribed than Cisplatin. However, Carboplatin still has significant toxicity and can incur drug resistance from repeat treatment. Recent trend in this field indicates that there may be a renewed interest in finding a significantly improved platinum drug. The third platinum drug, oxaliplatin, has been on European market for several years. Its efficacy appears to be lower than the current platinum drug, but it may have lower toxicity. Structures of cisplatin, carboplatin, and oxaliplatin are shown as follow:

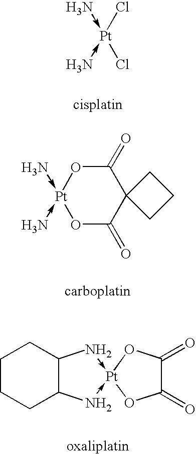

Problems Associated with Today's Anticancer Platinum Drugs

Cisplatin is known to function as an inhibitor to the DNA replication process; without the ability to replicate, cancer cells eventually die. It is believed that the inhibition is due to the intra-strand cross-linkage between Cisplatin and DNA through the two labile Pt—Cl binding sites, especially during the DNA replication process. However, Cisplatin is not very selective in attacking the cells. As Cisplatin destroys the cancerous cells, it can also cause certain damages to normal cells.

The most critical challenge for improving platinum drug is to significantly improve the therapeutic index (defined as the ratio of efficacy/toxicity). An ideal anticancer platinum drug should have a favorable therapeutic index. Unfortunately, a platinum drug works essentially by killing cancer cells through its cytotoxicity. Therefore, increasing the efficacy of such a drug is likely to increase the side effects due to the increased toxicity. This dilemma of the efficacy-toxicity parallel has hampered any significant improvement of platinum drugs for many years.

In order to significantly improve the performance of a platinum drug, the dilemma of efficacy-toxicity parallel must be addressed and solved. Therefore, the objects of this invention are to come up with a series of unique platinum compounds with the following improved characteristics in contract to current platinum drugs:

1. The efficacy must be substantially maintained.

2. The toxicity must be substantially reduced.

3. The modified efficacy and toxicity should result in a collective effect of improved therapeutic index, i.e., increased therapeutic index.

Other objects, features and advantages of the present invention will become apparent from the following detailed description. It should be understood, however, that the detailed description and the specific examples, while indicating preferred embodiments of the invention, are given by way of illustration only, since various changes and modifications wihin the spirit and scope of the invention will become apparent to those skilled in the art from this detailed description.

In our recently issued two patents (U.S. Pat. No. 6,534,096 issued Mar. 18, 2003; U.S. Pat. No. 6,548,541 issued Apr. 15, 2003) and a pending patent (No. 60/451,895, filed Mar. 4, 2003), methods of treating cancer using similar mechanism were disclosed. An example of the previous platinum compounds is shown as follows:

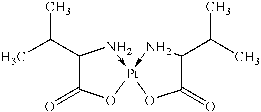

The present invention discloses a different series of platinum compounds, which can act in a similar dynamic “On-Off” mechanism, but are unique in their structures. To the best of our knowledge, no such compounds or use of such compounds for treating cancer has been disclosed yet.

### DETAILED DESCRIPTION OF THE INVENTION

This invention discloses a series of platinum compounds that are dynamic; they can be activated or deactivated favorably under different biological environments, thus, also called “On-Off” compounds herein. The important point is that this activation and deactivation mechanism is reversible and is a significant improvement over all current platinum drugs. Therefore, these platinum compounds can help resolve the parallel efficacy-toxicity dilemma.

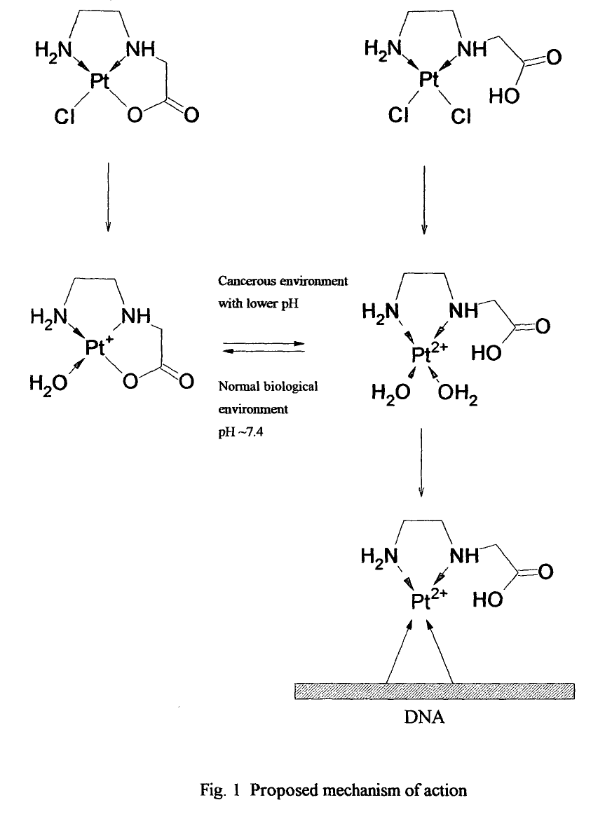

Due to the chemical equilibrium, under a normal biological environment with a pH of about 7.4, and under most cancerous environment with a pH lower than 7.4, the proportion of the “Off” compound and the “On” compound will be different. Relatively speaking, there will be fewer molecules in the “On” state under normal biological environment and more molecules in the “On” state under cancerous environment (see FIG. 1). Therefore, the overall toxicity to normal cells is less than that of any traditional platinum drugs.

Representative structures of the On-Off platinum compounds in the present invention are shown as follow:

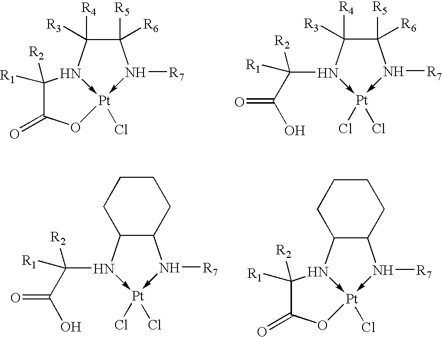

wherein each of R₁, R₂, R₃, R₄, R₅, R₆, and R₇, independently, is hydrogen, hydroxy, —CH₂—OH, —CH₂—CH₂—OH, —CH₂—CH₂—CH₂—OH, —CH₂—CH(CH₃)OH, —O—CH₃, —CH₂—CH₃, —O—CH₂—CH₂—CH₃, or lower alkyl; or each of CR₁R₂ (that is R₁ and R₂ together with the carbon attached), CR₃R₄, and CR₅R₆, independently, is C═O,

wherein “lower alkyl” means a linear, branched or cyclic hydrocarbon group containing from about 1 to 6 carbons, preferably from 1 to 3 carbons. Preferred lower alkyl groups include methyl, ethyl, and propyl.

To our best knowledge, none of the above platinum compounds were synthesized and disclosed as agents for cancer treatment.

### EXAMPLE 1

Synthesis of Compound I

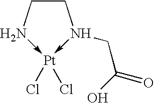

Compound I was synthesized according to the following method: In ca. 110 mL DI water, dissolve 709.2 mg (6.01 mmole) ethylenediamine-N-monoacetic acid (EDMA) and 2462.4 mg (5.93 nmole) K₂PtCl₄ at a mole ratio of 1:1. Mix well and heat at ca. 60° C. for 2 hours until the reddish solution becomes light yellow. Leave at room temperature for several days and collect the light yellow precipitate.

Melting point: 249.5-250° C.

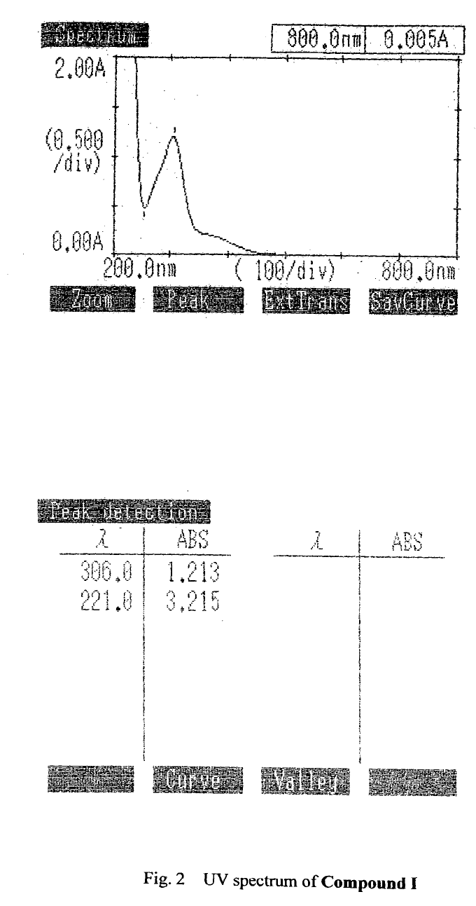

UV spectrum in water/methanol (1:1 v/v): λmax at 306 nm (FIG. 2)

CHN analysis: calcd. for C, 12.50; H, 2.60: N, 7.29. Found: C, 12.50; H, 2.59; N, 7.10. To our best knowledge, compound I has not been published.

### EXAMPLE 2

In-vitro Anticancer Activity of Compound I

Cell Line and Culture Conditions. The tumor cells were grown at 37° C. in a humidified atmosphere (5% CO₂) as monolayer cultures in RPMI 1640 medium supplemented with 10% FCS. Cells were trypsinized upon passage and maintained routinely. All cell lines were Mycoplasma free. The cell lines used was carcinoma of the uterus (UXF 1138L).

Test Solution. Compound I was dissolved in DI water as 4 mM stock solutions. The Pt-EDMA stock solution had a light yellow color and was transparent.

SRB assay. The SRB assay is routinely employed to measure cell proliferation by the US-NCI 60 cell line screen. Briefly, exponentially growing cells were harvested by trypsination, counted and seeded into 96well plates (2.000 cells/well). Compound was added in 8 concentrations ranging from 0.01-100 μM. Total protein mass was determined after 5 days of continuous drug exposure by addition of Sulforhodamine B (0.4%) solution. Extinctions were read at 515 nm and IC₅₀ values were calculated.

Results and Discussion

Compound I was evaluated for its antiproliferative activity in carcinoma of the uterus UXF 1138L and the resulted IC₅₀ was 3 μM.

To our best knowledge, none of the compounds disclosed in the present invention was previously disclosed as an anticancer treatment. The proposed mechanism of action of the compound are shown in FIG. 1.

Therefore, the present invention discloses a series of dynamic On-Off platinum compounds for treating cancer.

All platinum compounds disclosed in the present invention are dynamic, i.e., they can be transformed back and forth between the active form and non-active form. Depending on how acidic a biological environment is, different amount of the dynamic platinum compound can convert from non-active form to active form and vice versa. The more acidic the environment is, the more non-active form will be converted into active form. The microenvironment of a tumor can have a pH as low as 5.2 (Laurencot C. M. et.al., Oncol. Res, 7:371-379, 1995; Raghunand N. et. al., Biochem. Pharmacol., 57: 309-312, 1999.). It is apparent that the active form will be effective in killing cancer cells and the non-active form will not have much less cytotoxicity, thus, the side effects are significantly reduced.

As the active form binds onto DNA to inhibit the replication of cancer cells, its concentration decreases. This decreased concentration of the active form draws the equilibrium from the non-active form into the active form as shown in FIG. 1.

The present invention also discloses an improved method for treating cancer by administering the On-Off platinum compounds disclosed herein.

In a particular aspect, the present invention provides methods for the treatment of various malignancies. Treatment methods will involve treating an individual with a therapeutically effective amount of the platinum compounds in this invention, as described herein throughout. An effective amount is described, generally, as that amount sufficient to detectably and repeatedly to ameliorate, reduce, minimize or limit the extent of a disease or its symptoms. More rigorous definitions may apply, including elimination, eradication or cure of disease.

Because a variety of groups may be attached onto the backbone of the ligand (an example of the ligand is ethylenediamine-N-monoacetic acid) to form a variety of On-Off platinum compounds, these platinum(II) complexes can have significantly different physical properties (such as affinity, solubility, permeability, stereo effect, etc.) from those of cisplatin, carboplatin, or oxaliplatin. Therefore, it is conceivable that they would be useful in treating cancers that are not treated by cisplatin, carboplatin, or oxaliplatin.

The method discloses a method of treating cancer by said platinum compound wherein said cancer comprises cancer of the lung, brain, prostate, kidney, liver, ovary, endometrium, breast, skin, stomach, esophagus, head and neck, testicles, germ cancer, epithelial, colon, small intestine, thyroid, cervix, pancreas, glioblastoma, astrocytoma, oligodendroglioma, ependymomas, neurofibrosarcoma, meningia, lymphatic system, and blood.

The present invention discloses a pharmaceutical composition comprising:

i) a pharmaceutically acceptable amount of the platinum compound disclosed in this invention, and

ii) one or a plurality of pharmaceutically acceptable excipients.

The above pharmaceutically acceptable dosage form comprises between about 5 mg to about 1000 mg of the platinum compound. The therapeutic compositions of the present invention are advantageously administered in the form of injectable compositions either as liquid solutions or suspensions; solid forms suitable for solution in, or suspension in, liquid prior to injection may also be prepared. Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations, or powders. When the route is topical, the form may be a cream, ointment, salve, or spray.

The present invention discloses a method comprising administering to a cancer patient a therapeutically effective amount of the above pharmaceutical composition; the pharmaceutically acceptable form is administered once every one to six weeks and the regimen may be repeated until remission of said cancer is observed.

In the above method, the administration is oral or parenteral. In addition, the method further comprises treating said cancer patient with a further cancer treating agent. Said further cancer treating agent is a DNA damaging agent selected from the group consisting of verapamil, podophyllotoxin, procarbazine, mechlorethamine, cyclophospharmide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxol, transplatinum, 5-fluorouracil, vincristin, vinblastin, and methotrexate.

Said further cancer treating agent also comprises radiation, which is selected from the group consisting of X-ray radiation, UV-radiation, λ-radiation, or microwave radiation.

The method of treating cancer is effected by local delivery of said pharmaceutical composition, wherein said administering is effected by direct injection of a tumor in said cancer patient with said pharmaceutical composition.

The administration of said pharmaceutical composition may be effected endoscopically, intratracheally, intralesionally, percutaneously, intravenously, subcutaneously or intratumorally.

The method of treating cancer further comprises the step, prior to said administering, of resection of a tumor in said cancer patient.

The platinum compounds disclosed in the present invention may also be used in the treatment of AIDS (Acquired Immune Deficiency Syndrome). Because of the potential ability of these complexes to hamper the DNA or RNA replication process, it is conceivable that these complexes are effective against the HIV (Human Immunodeficiency Virus) and may be used for the treatment of AIDS. Because the platinum(II) ion may be camouflaged by a variety of ligands, theses platinum(II) complexes are less likely to cause the self defense of the HIV. Thus, these platinum(II) complexes may be used to treat AIDS.

Therefore, this invention also discloses a method of treating AIDS comprising administering orally or parenterally to an AIDS patient a therapeutically effective amount of a pharmaceutical composition comprising the platinum compound in the present invention.

Summary, Ramification, and Scope

In conclusion, a series of dynamic platinum compounds are disclosed in this invention. Also disclosed is a method of treating cancer comprising administering to a cancer patent said platinum compound.

Although the description above contains many specificities, these should not be construed as limiting the scope of the invention but as merely providing the illustrations of some of the presently preferred embodiments of this invention. Thus the scope of this invention should be determined by the appended claims and their legal equivalents, rather than by the examples given.

1. A compound comprising a formula as shown in the following structures: 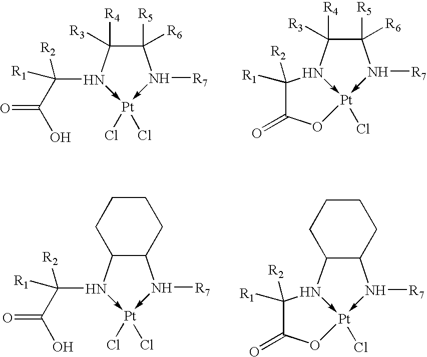
wherein each of R₁, R₂, R₃, R₄, R₅, R₆, and R₇, independently, is hydrogen, hydroxy, —CH₂—OH, —CH₂—CH₂—OH, —CH₂—CHOH—CH₃,—CH₂—CH₂—CH₂—OH, —CH₂—CHOH—CH₂—CH₃, —CH₂—CH(CH₃)OH, —O—CH₃, —O—CH₂—CH₃, —O—CH₂—CH₂—CH₃, —O—CH(CH₃)₂ or lower alkyl; or each of CR₁R₂ (that is R₁ and R₂ together with the carbon attached), CR₃R₄, and CR₅R₆, independently, is C═O,
wherein “lower alkyl” means a linear, branched or cyclic hydrocarbon group containing from about 1 to 6 carbons, preferably from 1 to 3 carbons. Preferred lower alkyl groups include methyl, ethyl, and propyl.

2. A method of treating cancer by administering to a cancer patient a therapeutically effective amount of the compound in claim 1.

3. The method of claim 2 wherein said cancer comprises cancer of the lung, brain, prostate, kidney, liver, ovary, endometrium, breast, skin, stomach, esophagus, head and neck, testicles, germ cancer, epithelial, colon, small intestine, thyroid, cervix, pancreas, glioblastoma, astrocytoma, oligodendroglioma, ependymomas, neurofibrosarcoma, meningia, lymphatic system, and blood.

4. A pharmaceutical composition comprising:
i) a pharmaceutically acceptable amount of the platinum compound in claim 1, and
ii) one or a plurality of pharmaceutically acceptable excipients.

5. A method of treating cancer comprising administering to a cancer patient a therapeutically effective amount of the pharmaceutical composition in claim 4

6. The method of claim 5 wherein said cancer comprises cancer of the lung, brain, prostate, kidney, liver, ovary, endometrium, breast, skin, stomach, esophagus, head and neck, testicles, germ cancer, epithelial, colon, small intestine, thyroid, cervix, pancreas, glioblastoma, astrocytoma, oligodendroglioma, ependymomas, neurofibrosarcoma, meningia, lymphatic system, and blood.

7. The method of claim 5 wherein said administration is oral.

8. The method of claim 5 wherein said administration is parenteral.

9. The method of claim 5 wherein said method further comprises treating said cancer patient with a further cancer treating agent.

10. The method of claim 9, wherein said further cancer treating agent is a DNA damaging agent selected from the group consisting of verapamil, podophyllotoxin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxol, transplatinum, 5-fluorouracil, vincristin, vinblastin and methotrexate.

11. The method of claim 5 wherein said method further comprises treating said cancer patient with a radiation.

12. The method of claim 11, wherein said radiation is selected from the group consisting of X-ray radiation, UV-radiation, γ-radiation, or microwave radiation.

13. The method of claim 5, wherein said administering is effected by local delivery of said pharmaceutical composition.

14. The method of claim 5, wherein said administering is effected by direct injection of a tumor in said cancer patient with said pharmaceutical composition.

15. The method of claim 5, wherein said administering comprises delivering said pharmaceutical composition endoscopically, intratracheally, intralesionally, percutaneously, intravenously, subcutaneously or intratumorally.

16. The method of claim 5, further comprising the step, prior to said administering, of resection of a tumor in said cancer patient.

17. A method of treating Acquired Immune Deficiency Syndrome (AIDS) comprising administering orally or parenterally to an AIDS patient a therapeutically effective amount of a pharmaceutical composition comprising the compound in claim 1.
